(19) United States
(12) Patent Application Publication
(10) Pub. No.: US 2011/0071479 A1
(43) Pub. Date: Mar. 24, 2011

(54) Blood Vessel Access Instrument with Vessel Entry Indicator

(75) Inventors: Steven A. Holley, West Roxbury, MA (US); Bradley M. Stewart, Trevor, WI (US)

(73) Assignee: Tyco Healthcare Group LP, Mansfield, MA (US)

(21) Appl. No.: 12/564,976

(22) Filed: Sep. 23, 2009

(51) Int. Cl. A61M 5/32; A61M 25/00

(52) U.S. Cl. 604/272; 604/264

(57) ABSTRACT

A surgical instrument for administering fluids and having a vessel entry indicator to confirm proper placement of the surgical instrument within the vessel includes a vessel access member adapted for accessing a blood vessel and having insertion and trailing ends. The access member defines a primary lumen for administering fluids to the blood vessel and a secondary lumen. A pressure detector is in fluid communication with the secondary lumen. The pressure detector is adapted to detect pressure associated with a disposition of the insertion end of the access member in the blood vessel. The access member may define a port adjacent the leading end and in fluid communication with the secondary lumen. In one embodiment, the secondary lumen is independent of the primary lumen. The pressure detector may include a float valve responsive to pressure of blood entering from the blood vessel through the port and into the secondary lumen. In the alternative, the pressure detector includes a pressure gauge. The pressure gauge is adapted to register pressure associated with a presence of blood entering from the blood vessel through the port and into the secondary lumen.

### BACKGROUND

1. Technical Field

The present disclosure relates to a surgical instrument for accessing a blood vessel, and, in particular, relates to a surgical access instrument incorporating a vessel entry indicator to confirm placement of the access instrument within the blood vessel during administration of fluids.

2. Background of Related Art

Surgical access instruments including intravenous (IV) needles or cannulas are employed during the administration of therapeutic fluids into the blood vessel in conjunction with intravenous (IV) procedures. Such IV procedures may include intermittent or continuous IV therapy for the introduction of specialty pharmaceuticals, blood transfusions, chemotherapy regimens, antibiotic therapy, parenteral nutrition, dehydration treatment or the like. During an intravenous procedure, it is imperative that the IV needle remain within the blood vessel and not become inadvertently dislodged or lose access to the blood vessel. For example, during the IV administration of chemotherapy drugs, if the IV needle is dislodged or removed for any reason from the vein, the drugs may enter the subcutaneous tissue and cause damage to the tissue subjected to the drugs.

### SUMMARY

Accordingly, the present disclosure is directed to a surgical instrument for administering fluids and having a vessel entry indicator to confirm proper placement of the surgical instrument within the vessel. The surgical instrument includes a vessel access member adapted for accessing a blood vessel and having insertion and trailing ends. The access member defines a primary lumen for administering fluids to the blood vessel and a secondary lumen. A pressure detector is in fluid communication with the secondary lumen. The pressure detector is adapted to detect pressure associated with a disposition of the insertion end of the access member in the blood vessel. The access member may define a port adjacent the leading end and in fluid communication with the secondary lumen. In one embodiment, the secondary lumen is independent of the primary lumen. The pressure detector may include a float valve responsive to pressure of blood entering from the blood vessel through the port and into the secondary lumen. In the alternative, the pressure detector includes a pressure gauge. The pressure gauge is adapted to register pressure associated with a presence of blood entering from the blood vessel through the port and into the secondary lumen.

In another embodiment, the secondary lumen is in fluid communication with the primary lumen whereby pressure is detected by the pressure detector upon discontinuance of administering fluids through the primary lumen, to thereby permit blood to pass through the primary lumen and enter the secondary lumen. The pressure detector in this embodiment may be a pressure gauge.

The primary lumen and the secondary lumen may be coaxially arranged about a reference longitudinal axis defined by the access member. Alternatively, the primary lumen and the secondary lumen are offset with respect to a reference longitudinal axis defined by the access member. The access member may include a housing and an elongate member extending from the housing.

A source of therapeutic fluids is adapted to be coupled to the primary lumen.

In another embodiment, the surgical instrument for administering fluids includes a vessel access member adapted for accessing a blood vessel and having insertion and trailing ends. The access member defines a primary lumen for administering fluids to the blood vessel and a secondary lumen. The access member has an inlet port adjacent the leading end and in fluid communication with the secondary lumen to permit blood to pass from the blood vessel to enter the secondary lumen, and a return port displaced from the inlet port and in fluid communication with the primary lumen to permit blood to pass from the secondary lumen to the primary lumen for return with the administering fluids to the blood vessel. A fluid flow detector is in fluid communication with the secondary lumen. The flow detector is adapted to detect passage of blood through the secondary lumen when the insertion end of the access member is disposed within the blood vessel. The fluid flow detector may be a flow gauge. The primary lumen and the secondary lumen may be coaxially arranged about a reference longitudinal axis defined by the access member. Alternatively, the primary lumen and the secondary lumen may be offset with respect to a reference longitudinal axis defined by the access member.

### BRIEF DESCRIPTION OF THE DRAWINGS

The accompanying drawings, which are incorporated in and constitute a part of this specification, illustrate embodiments of the present disclosure and, together with the detailed description of the embodiments given below, serve to explain the principles of the disclosure, wherein:

FIG. 1 is a side view of a system for administering fluids in accordance with the principles of the present disclosure illustrating the access instrument coupled to a syringe;

FIG. 2 is a side cross-sectional view of the access instrument of the system illustrating the housing and the elongate member extending from the housing;

FIG. 3 is a cross-sectional view taken along the lines 3-3 of FIG. 1 illustrating the coaxial arrangement of the primary lumen and the secondary lumens within the elongate member;

FIG. 4 is a cross-sectional view illustrating an alternate offset arrangement of the primary lumen and the secondary lumen;

FIG. 5 is a side cross-sectional view of an alternate embodiment of the access instrument; and

FIG. 6 is a side cross-sectional view of another alternate embodiment of the access instrument.

### DETAILED DESCRIPTION OF PREFERRED EMBODIMENTS

The attached figures illustrate exemplary embodiments of the present disclosure and are referenced to describe the embodiments depicted therein. Hereinafter, the disclosure will be described in detail by explaining the figures wherein like reference numerals represent like parts throughout the several views.

The exemplary embodiments of the apparatus disclosed herein are discussed in terms of performing a therapeutic procedure involving administration of fluids into a blood vessel of the subject. Such therapeutic procedures are inclusive of but, not limited to, intermittent or continuous IV procedures for the introduction of specialty pharmaceuticals, blood transfusions, chemotherapy regimens, antibiotic therapy, parenteral nutrition, dehydration treatment or the like. However, it is envisioned that the present disclosure may be employed with many applications and related treatments of diseases and body ailments of a subject.

In the following discussion, the term “subject” refers to a human patient or other animal. The term “clinician” refers to a doctor, nurse, or other care provider and may include support personnel. The term “trailing” or “proximal” refers to the portion of the instrument closest to the operator while the term “entry” or “distal” refers to the portion of the instrument remote from the operator.

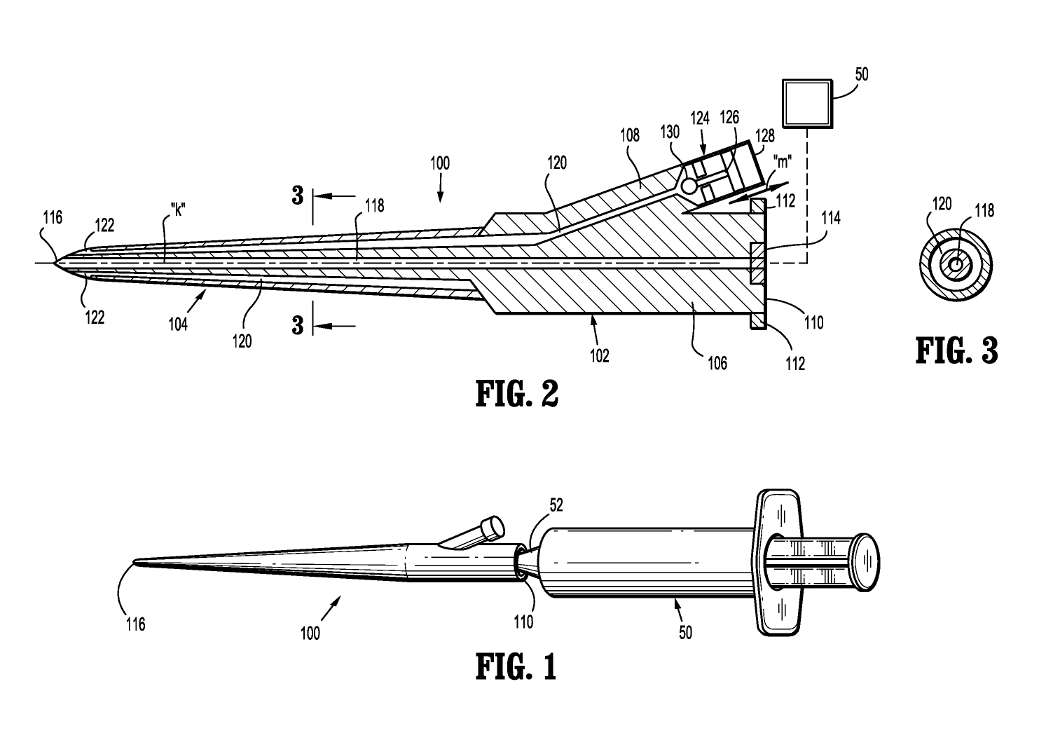

Referring now to FIG. 1, the surgical system in accordance with the principles of the present disclosure is illustrated. System 10 is intended to administer fluids in conjunction with a peripheral intravenous (PIV) procedure. System 10 includes IV fluid source 50 and access instrument 100 which administers the fluids of the IV fluid source 50. The IV fluid source 50 may be in the form of a syringe as shown in FIG. 1 or may be an external IV fluid source such as an IV bag, drip or the like. The external IV fluid source 50 is couplable to access instrument 100 through conventional coupling means including, e.g., various commercially available tubings, lines and couplings, which are adapted to connect to access instrument 100.

Referring now to FIG. 2, in conjunction with FIG. 1, access instrument 100 will be discussed. Access instrument 100 is an access member such as a needle, cannula or catheter adapted to access a blood vessel of the subject, particularly, in connection with peripheral IV procedures where the arm or hands are accessed to deliver IV fluids. Access instrument 100 also may be adapted to access a vein in the leg or foot or, in the case of an infant, the scalp. Access instrument 100 includes housing 102 and elongate member 104 connected to the housing 102 and extending therefrom. Housing 102 is adapted to be grasped by the clinician and may be formed of any suitable metal or polymeric material. Housing 102 may be clear or opaque Housing 102 includes main body 106 and detector leg 108 which extends from the main body 106 in oblique relation to the longitudinal axis “k” of the housing 102. Main body 106 defines proximal or trailing end 110 which is couplable to IV fluid source 50 through various conventional coupling means. In FIG. 2, IV fluid source 50 is represented schematically and may be an IV bag, drip or the like. Exemplative coupling means for coupling main body 106 to external IV source 50 include luer connectors, bayonet couplings or the like. In FIG. 2, bayonet lugs 112 are shown. As appreciated, the coupling means on the IV line connected to IV fluid source 50 would incorporate corresponding structure to cooperate with the coupling means of main body 106 such as a bayonet female receptor with matching slots for the male bayonet lugs 112. In the situation where fluid source 50 is a syringe (FIG. 1), leading end 52 of the syringe 50 may penetrate or pass through internal seal 114 within trailing end 110 of main body 106 to be releasably secured therein. Internal seal 114 is adapted to form a substantial fluid tight seal about syringe leading end 52 and may be a septum seal or zero closure seal fabricated from a suitable elastomeric material.

Elongate member 104 is preferably formed of a suitable biocompatible material such as stainless steel or other polymeric materials. Elongate member 104 may be clear or opaque. The diameter of elongate member 104 may vary, but, typically ranges from a 12-gauge to a 26 gauge size, e.g., cannula. Elongate member 104 defines leading end 116 which may be adapted to penetrate, incise or pass through tissue to access the vein.

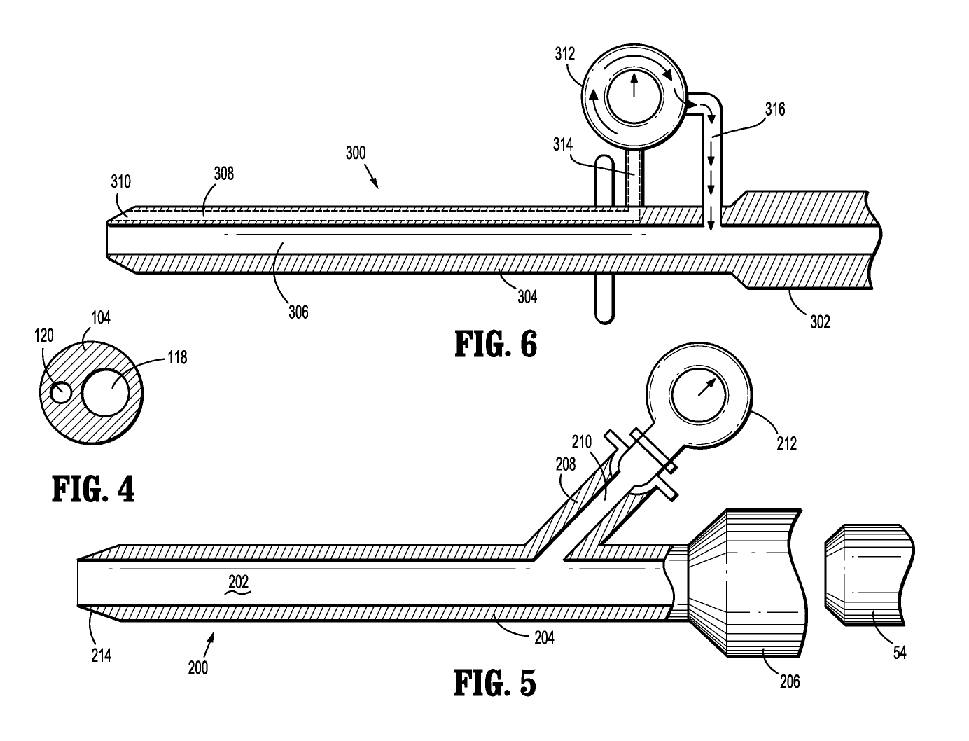

With reference to FIGS. 1-3, access instrument 100 defines primary lumen 118 which extends from trailing end 110 of housing 102 through leading end 116 of elongate member 104. Primary lumen 118 is in fluid communication with IV fluid source 50 to deliver or administer the IV fluids to the subject's vein. Access instrument 100 further defines secondary lumen 120 which extends through inflow ports 122 adjacent entry end 116 of elongate member 104 through the elongate member 104 and detector leg 108. Secondary lumen 120 is intended to permit blood to flow from the vein into elongate member 104 and detector leg 108 for reasons to be hereinbelow discussed. Inflow ports 122 may be in diametrical opposed relation and formed in the wall of elongate member 104. Alternatively, inflow port 122 may be an axial port having an annular configuration. Primary and secondary lumens may be concentrically or coaxially arranged about the longitudinal axis as shown in FIG. 3. In the alternative, primary and secondary lumens 118, 120 may be offset with respect to each other as depicted in FIG. 4.

Referring now to FIG. 2, access instrument 100 further includes pressure detector 124 mounted to detector leg 108 of housing 102 for sensing the pressure within secondary lumen 120. The pressure is associated with the presence of blood when entry end 116 of elongate member 104 is disposed within vein. Pressure detector 124, in one embodiment, includes float valve 126 which is mounted within valve housing 128. Float valve 126 is adapted to reciprocate within housing, in the direction of directional arrows “m”, responsive to the presence or lack of presence of blood within secondary lumen 120. Float valve 126 may be any conventional float valve suitable for this intended purpose including [Inventor, please provide examples]. In one embodiment, float valve 126 is adapted to reciprocate due to the increased air pressure within secondary lumen due to the presence of blood. In an alternate embodiment, float valve 126 incorporates a floating ball 130 which floats relative to the blood when the blood communicates through the secondary lumen 120 and within valve housing 128. Various other detectors are envisioned including gauges, transducers, fiber optic sensors, mechanical spring sensors, piezoresistive sensors or transducers, and sensors based upon changes in measured capacitance.

In use, entry end 116 of elongate member 104 is advanced through tissue to access the desired vein. When entry end 116 is positioned in the vein, blood enters through inflow port 122 to pass within secondary lumen 120. The presence of blood within secondary lumen 120 is detected by float valve 126 which moves in a proximal direction within valve housing 128. Movement of float valve 126 in the proximal direction is confirmed by the clinician who may view the float valve 126 through a transparent portion of valve housing 128. Access instrument 100 may then be connected to the fluid source 50 to permit administration of the fluids and commencement of therapy. Throughout the fluid administrating process, the clinician will monitor float valve 126 and determine if the float valve 126 is activated and/or oscillating (corresponding to the rhythm of the pulse) which is indicative of the access instrument 100 being lodged appropriately within the vein.

FIG. 5 illustrates an alternate embodiment of the present disclosure. In accordance with this embodiment, access instrument 200 includes primary lumen 202 extending the length of elongate member 204 and the length of housing 206. Access instrument 200 further includes offset detector leg 208 which is arranged in oblique relation to the longitudinal axis of access instrument. Offset leg 208 defines secondary lumen 210 which is in fluid communication with primary lumen 202. Offset leg 208 further includes pressure gauge 212 mounted thereto. Any suitable commercially available pressure gauge may be employed. Pressure gauge 212 is adapted to monitor pressure within secondary lumen 210 due to the presence of blood within the secondary lumen 210. In one embodiment, offset leg 208 includes a male/female luer connector which connects to a corresponding connector of pressure gauge 212. In FIG. 5, IV connector 54 for connecting external IV fluid source 50 to housing 206 is shown.

In use, primary lumen 202 is flushed with a saline. Access instrument 200 is then advanced within the tissue and into the vein with a blood flashback flowing through primary and/or secondary lumens 202, 210 confirming that entry end 214 is within the vein. The presence of blood within primary and/or secondary lumens 202, 210 may be visualized through a transparent region of elongate member 204 or housing 206. Housing 206 of access instrument 200 is connected to IV fluid connector 54 or a syringe, and administration of fluids through primary lumen 202 is commenced. When it is desired to check that entry end 214 of elongate member 204 is properly positioned within the vein, the infusion or administration of fluids is interrupted. The blood is permitted to communicate through primary lumen 202 and into secondary lumen 210. Pressure associated with the presence of blood is registered by pressure gauge 212 either through a constant pressure or through an oscillation of pressure (corresponding to the subject's pulse). If no pressure is recorded by pressure gauge 212, it can be determined that access instrument 200 is not within the vein, thus, prompting the clinician to reposition entry end 214 of the access instrument 200 within the vein.

FIG. 6 illustrates another alternate embodiment of the present disclosure. Access instrument 300 includes housing 302 and elongate member 304 extending from the housing 302. Access instrument 300 includes primary lumen 306 extending the length of the access instrument 300 for administration of fluids from a fluid source. Elongate member 304 further includes secondary lumen 308 within the outer wall of elongate member 304 and outlet port 310 in fluid communication with the secondary lumen 308. Access instrument 300 further includes flow gauge 312 mounted to elongate member 304. Flow gauge 312 may be any suitable conventional gauge adapted to detect the presence of fluid or detect the flow of fluid. Flow gauge 312 is in fluid communication with primary lumen through inflow tube 314 and outflow tube 316. Flow gauge 312 may be mounted to housing 302.

In use, entry end 310 of access instrument 300 is positioned within the vein. Administration of fluids from the fluid source (not shown) through primary lumen 306 is commenced. Concurrently with the administration of fluids, blood enters secondary lumen 308 through entry port 310 and communicates through inflow tube 314 and travels across flow gauge 312. This flow of blood is registered by flow gauge 312. The blood thereafter communicates through outflow tube 316 where it is released into primary lumen 300 for return, along with the IV fluids, into the vein of the subject. If no fluid flow is registered by flow gauge 312, the clinician will determine that access instrument 300 is not properly positioned within the vein, thus, prompting the clinician to act accordingly to reposition the access instrument 300.

Although the foregoing disclosure has been described in some detail by way of illustration and example, for purposes of clarity or understanding, it will be obvious that certain changes and modifications may be practiced within the scope of the appended claims.

What is claimed is:

1. A surgical instrument for administering fluids, which comprises:
a vessel access member adapted for accessing a blood vessel and having insertion and trailing ends, the access member defining a primary lumen for administering fluids to the blood vessel and a secondary lumen; and
a pressure detector in fluid communication with the secondary lumen, the pressure detector adapted to detect pressure associated with a disposition of the insertion end of the access member in the blood vessel.

2. The surgical instrument according to claim 1 wherein the access member defines a port adjacent the leading end and in fluid communication with the secondary lumen.

3. The surgical instrument according to claim 1 wherein the secondary lumen is independent from the primary lumen.

4. The surgical instrument according to claim 3 wherein the pressure detector includes a float valve, the float valve responsive to pressure of blood entering from the blood vessel through the port and into the secondary lumen.

5. The surgical instrument according to claim 3 wherein the pressure detector includes a pressure gauge, the pressure gauge adapted to register pressure associated with a presence of blood entering from the blood vessel through the port and into the secondary lumen.

6. The surgical instrument according to claim 1 wherein the secondary lumen is in fluid communication with the primary lumen whereby pressure is detected by the pressure detector upon discontinuance of administering fluids through the primary lumen, to thereby permit blood to pass through the primary lumen and enter the secondary lumen.

7. The surgical instrument according to claim 6 wherein the pressure detector is a pressure gauge.

8. The surgical instrument according to claim 1 wherein the primary lumen and the secondary lumen are coaxially arranged about a reference longitudinal axis defined by the access member.

9. The surgical instrument according to claim 1 wherein the primary lumen and the secondary lumen are offset with respect to a reference longitudinal axis defined by the access member.

10. The surgical instrument according to claim 1 wherein the access member includes a housing and an elongate member extending from the housing.

11. The surgical instrument according to claim 10 including a source of therapeutic fluids adapted to be coupled to the primary lumen.

12. The surgical instrument according to claim 10 wherein the leading end is associated with the elongate member, the leading end being adapted to penetrate tissue.

13. A surgical instrument for administering fluids, which comprises:
a vessel access member adapted for accessing a blood vessel and having insertion and trailing ends, the access member defining a primary lumen for administering fluids to the blood vessel and a secondary lumen, the access member having an inlet port adjacent the leading end and in fluid communication with the secondary lumen to permit blood to pass from the blood vessel to enter the secondary lumen, and a return port displaced from the inlet port and in fluid communication with the primary lumen to permit blood to pass from the secondary lumen to the primary lumen for return with the administering fluids to the blood vessel; and
a fluid flow detector in fluid communication with the secondary lumen, the flow detector adapted to detect passage of blood through the secondary lumen when the insertion end of the access member is disposed within the blood vessel.

14. The surgical instrument according to claim 13 wherein the fluid flow detector is a flow gauge.

15. The surgical instrument according to claim 13 wherein the primary lumen and the secondary lumen are coaxially arranged about a reference longitudinal axis defined by the access member.

16. The surgical instrument according to claim 13 wherein the primary lumen and the secondary lumen are offset with respect to a reference longitudinal axis defined by the access member.
